(19) United States
(12) Patent Application Publication
(10) Pub. No.: US 2022/0339033 A1
(43) Pub. Date: Oct. 27, 2022

(54) COUPLING A FLUID-DYNAMICS CARTRIDGE WITH A PHACOEMULSIFIER PROBE BODY

(71) Applicant: JOHNSON & JOHNSON SURGICAL VISION, INC., Santa Ana, CA (US)

(72) Inventors: Yehuda Algawi, Binyamina (IL); Assaf Govari, Haifa (IL); Eran Aharon, Haifa (IL); Ilya Sitnitsky, Nahariya (IL)

(21) Appl. No.: 17/375,614

(22) Filed: Jul. 14, 2021

Related U.S. Application Data
(63) Continuation-in-part of application No. 17/240,505, filed on Apr. 26, 2021.

(51) Int. Cl. A61F 9/007

(52) U.S. Cl. CPC A61F 9/00745

(57) ABSTRACT

A medical probe includes a probe body shaped to define a distal section of a fluid channel, a cartridge, which is shaped to define a proximal section of the fluid channel and comprises a valve configured to regulate flow of a fluid through the proximal section of the fluid channel, and a clip configured to reversibly couple the cartridge with the probe body by sliding over the probe body and the cartridge while the cartridge contacts the probe body such that the proximal section of the fluid channel is in fluidic communication with the distal section of the fluid channel. Other embodiments are also described.

### CROSS-REFERENCE TO RELATED APPLICATIONS

The present application is a continuation-in-part of U.S. application Ser. No. 17/240,505, entitled “Solenoid valve shock absorber,” filed Apr. 26, 2021, which is hereby incorporated by reference in its entirety.

### FIELD OF THE INVENTION

The present invention is related generally to the field of eye surgery, and particularly to phacoemulsification procedures.

### BACKGROUND

During a phacoemulsification procedure, the lens of an eye is emulsified by ultrasonic waves.

US Patent Application Publication 2021/0000648 describes an ophthalmic surgical instrument including an instrument hand grip portion having an elongated configuration with proximal and distal ends, and a tip portion extending from the distal end. The tip portion has the form of an elastomeric element having a base secured to the distal end of the hand grip portion and a free end extending from the base. The free end is configured for massaging the trabecular meshwork and outer wall of the canal of Schlemm in the eye to improve aqueous outflow for the reduction of intraocular pressure.

US Patent Application Publication 2020/0383833 describes a medical device for removing lens tissue from inside a capsular bag of an eye including a cam assembly operatively coupled to a vacuum generation source positioned within the housing. A first portion is operatively coupled to the vacuum generation source and a second portion is operatively coupled to the first portion and to the shaft. The first portion is capable of rotating about an axis to cause the vacuum generation source to generate vacuum through the lumen. The second portion is capable of rotating about the axis with the first portion to cause the shaft to oscillate. A first degree of actuation of a trigger causes the vacuum generation source to generate vacuum within the lumen of the shaft, and a second degree of actuation of the trigger causes the shaft to oscillate as the second portion rotates. Related systems, devices, and methods are provided.

### SUMMARY OF THE INVENTION

There is provided, in accordance with some embodiments of the present invention, a medical probe including a probe body shaped to define a distal section of a fluid channel, a cartridge, which is shaped to define a proximal section of the fluid channel and includes a valve configured to regulate flow of a fluid through the proximal section of the fluid channel, and a clip configured to reversibly couple the cartridge with the probe body by sliding over the probe body and the cartridge while the cartridge contacts the probe body such that the proximal section of the fluid channel is in fluidic communication with the distal section of the fluid channel.

In some embodiments, the clip is U-shaped.

In some embodiments, the probe body includes one or more electrical interfaces configured to transfer electricity to the cartridge while the cartridge is coupled with the probe body.

In some embodiments, the clip is slidably disposed around the probe body.

In some embodiments, an outer surface of the cartridge is shaped to define one or more ridges, an inner surface of the clip is shaped to one or more protrusions, and the clip is configured to couple the cartridge with the probe body by sliding over the probe body and the cartridge until the protrusions are aligned with the ridges.

In some embodiments, the probe body and clip are configured to facilitate holding the clip, alternatingly, in an unlocked position, in which the protrusions are misaligned with the ridges, and a locked position, in which the protrusions are aligned with the ridges.

In some embodiments, the protrusions are first protrusions, the inner surface of the clip is further shaped to define one or more second protrusions, the probe body is shaped to define one or more unlocked-state indentations and one or more locked-state indentations, and the second protrusions are configured to snap into the unlocked-state indentations, respectively, upon the clip reaching the unlocked position, and to snap into the locked-state indentations, respectively, upon the clip reaching the locked position.

In some embodiments, the clip includes at least one spring, the probe body is shaped to define one or more unlocked-state indentations and one or more locked-state indentations, and the spring is configured to engage with the unlocked-state indentations upon the clip reaching the unlocked position, and to engage with the locked-state indentations upon the clip reaching the locked position.

In some embodiments, a face of the clip is shaped to define one or more indentations, and the probe body is shaped to define one or more pockets, and includes: one or more pins, each of the pins including a front pin-portion disposed within a different respective one of the indentations and a back pin-portion disposed within a different respective one of the pockets; and one or more springs, each of the springs being coupled at a front spring-end to a different respective one of the pins and at a back spring-end to an inside of a different respective one of the pockets, the springs and pins being configured to rotate within the pockets as the clip slides between the unlocked position and the locked position.

In some embodiments, the clip is slidably disposed around the cartridge.

In some embodiments, an outer surface of the probe body is shaped to define one or more ridges, an inner surface of the clip is shaped to one or more protrusions, and the clip is configured to couple the cartridge with the probe body by sliding over the probe body and the cartridge until the protrusions are aligned with the ridges, respectively.

In some embodiments, the cartridge and clip are configured to facilitate holding the clip, alternatingly, in an unlocked position, in which the protrusions are misaligned with the ridges, and a locked position, in which the protrusions are aligned with the ridges.

In some embodiments, the protrusions are first protrusions, the inner surface of the clip is further shaped to define one or more second protrusions, the cartridge is shaped to define one or more unlocked-state indentations and one or more locked-state indentations, and the second protrusions are configured to snap into the unlocked-state indentations, respectively, upon the clip reaching the unlocked position, and to snap into the locked-state indentations, respectively, upon the clip reaching the locked position.

In some embodiments, the clip includes at least one spring, the cartridge is shaped to define one or more unlocked-state indentations and one or more locked-state indentations, and the spring is configured to engage with the unlocked-state indentations upon the clip reaching the unlocked position, and to engage with the locked-state indentations upon the clip reaching the locked position.

In some embodiments, a face of the clip is shaped to define one or more indentations, and the cartridge is shaped to define one or more pockets, and includes: one or more pins, each of the pins including a front pin-portion disposed within a different respective one of the indentations and a back pin-portion disposed within a different respective one of the pockets; and one or more springs, each of the springs being coupled at a front spring-end with a different respective one of the pins and at a back spring-end with an inside of a different respective one of the pockets, the springs and pins being configured to rotate within the pockets as the clip slides between the unlocked position and the locked position.

There is further provided, in accordance with some embodiments of the present invention, a method including bringing a cartridge, which is shaped to define a proximal section of a fluid channel and includes a valve configured to regulate flow of a fluid through the proximal section of the fluid channel, into contact with a probe body of a medical probe, the probe body being shaped to define a distal section of the fluid channel, such that the proximal section of the fluid channel is in fluidic communication with the distal section of the fluid channel. The method further includes reversibly coupling the cartridge with the probe body, by sliding a clip over the probe body and the cartridge while the cartridge contacts the probe body.

The present invention will be more fully understood from the following detailed description of embodiments thereof, taken together with the drawings, in which:

### BRIEF DESCRIPTION OF THE DRAWINGS

FIG. 1 is a partly pictorial, partly block diagram view of a phacoemulsification system constructed and operative in accordance with some embodiments of the present invention;

FIGS. 2A-B are schematic illustrations of a phacoemulsification probe, in accordance with some embodiments of the present invention;

FIG. 3A is a schematic view of an interior of a fluid-dynamics cartridge, in accordance with some embodiments of the present invention;

FIGS. 3B-C are cross-sections through a fluid-dynamics cartridge, in accordance with some embodiments of the present invention;

FIG. 4 is a schematic illustration of a probe body with a fluid-dynamics cartridge and clip, in accordance with some embodiments of the present invention;

FIG. 5 is a schematic illustration of a clip in an unlocked position, in accordance with some embodiments of the present invention;

FIG. 6 is a schematic illustration of the proximal end of a probe body with a clip, in accordance with some embodiments of the present invention;

FIG. 7A is a schematic illustration of a clip, in accordance with some embodiments of the present invention;

FIG. 7B is a schematic illustration of the proximal end of a probe body, in accordance with some embodiments of the present invention;

FIG. 7C is a schematic illustration of a clip in an unlocked position, in accordance with some embodiments of the present invention; and

FIG. 7D is a schematic illustration of a clip in a locked position, in accordance with some embodiments of the present invention.

### DETAILED DESCRIPTION OF EMBODIMENTS

### Overview

Embodiments of the present invention include various mechanisms for reversibly coupling a fluid-dynamics cartridge with the body of a phacoemulsification probe. Advantageously, the fluid-dynamics cartridge helps regulate the flow of fluid to and from the distal end of the probe.

First, the cartridge is brought into contact with the proximal end of the probe body such that the cartridge is in fluidic communication with the probe body. Subsequently, to lock the cartridge in place, a clip is slid over the proximal end of the probe body together with the cartridge, until the clip reaches a “locked position” in which the clip inhibits separation of the cartridge from the probe body. Conversely, to unlock the cartridge, the clip is slid in the opposite direction, until the clip reaches an “unlocked position” in which the clip does not inhibit separation of the cartridge.

Advantageously, various features of the probe body and/or clip may facilitate holding the clip in the unlocked or locked position. Moreover, as the clip reaches either one of the positions, these features may provide tactile and/or auditory feedback indicating that the clip has reached the position. For example:

(i) In some embodiments, the proximal end of the probe body is shaped to define at least one pair of indentations, and the clip is shaped to define at least one protrusion. Upon the clip reaching the locked position, the protrusion snaps into one of the indentations. Conversely, upon the clip reaching the unlocked position, the protrusion snaps into the other indentation.

(ii) In other embodiments, instead of a protrusion, the clip comprises a spring. Upon the clip reaching the locked position, the spring springs into one of the indentations in the proximal end of the probe body. Conversely, upon the clip reaching the unlocked position, the spring springs into the other indentation.

(iii) In yet other embodiments, the distal face of the clip is shaped to define at least one indentation, and the proximal end of the probe body is shaped to define at least one pocket. The probe body comprises a pin having a front portion disposed within the indentation and a back portion disposed within the pocket. The probe body further comprises a spring coupled at one end to the pin and at the other end to the inside of the pocket. As the clip slides between the unlocked position and the locked position, the spring and pin rotate within the pocket. The spring helps hold the clip in the unlocked or locked position, in that movement of the clip from one position to the other cannot occur without compression of the spring. Moreover, the expansion of the spring, and/or a sound produced as the spring contacts the wall of the pocket, may indicate to the user that the clip has reached the unlocked or locked position.

### System Description

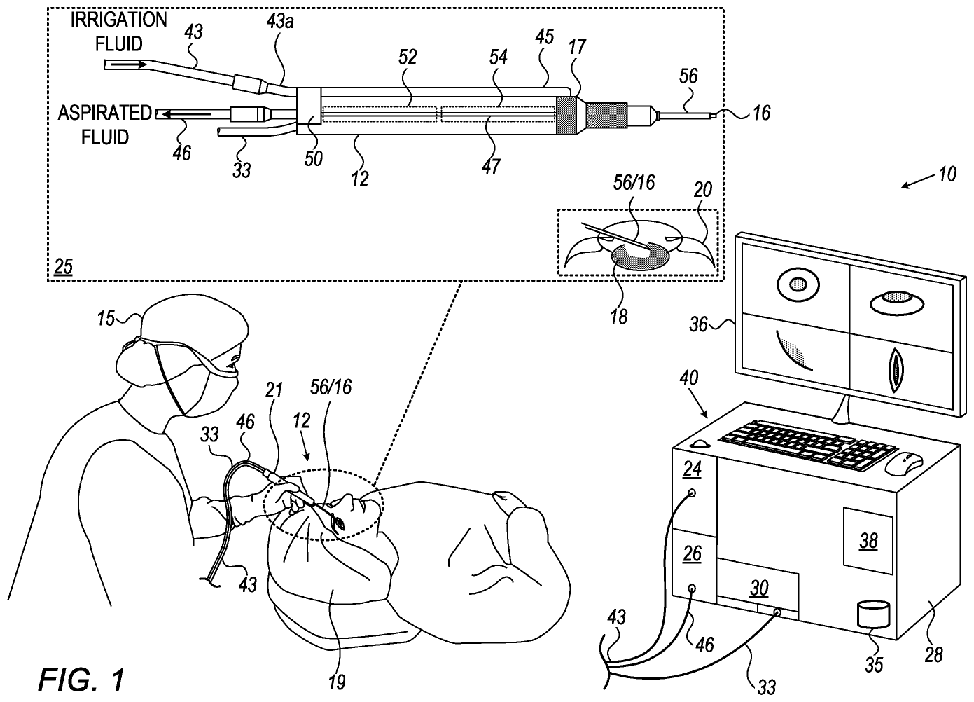

Reference is initially made to FIG. 1, which is a partly pictorial, partly block diagram view of a phacoemulsification system 10 constructed and operative in accordance with some embodiments of the present invention.

Phacoemulsification system 10 comprises a handheld phacoemulsification probe 12. As seen in the pictorial view of phacoemulsification system 10, and in inset 25, phacoemulsification probe 12 comprises a needle 16, a probe body 17, and a coaxial irrigation sleeve 56 that at least partially surrounds needle 16 and creates a fluid pathway between the external wall of the needle and the internal wall of the irrigation sleeve, where needle 16 is hollow to provide an aspiration channel. Moreover, irrigation sleeve 56 may have one or more side ports at, or near, the distal end to allow irrigation fluid to flow towards the distal end of phacoemulsification probe 12 through the fluid pathway and out of the port(s).

Needle 16 is configured for insertion into a lens capsule 18 of an eye 20 of a patient 19 by a physician 15 to remove a cataract. While needle 16 (and irrigation sleeve 56) are shown in inset 25 as a straight object, any suitable needle may be used with phacoemulsification probe 12, such as a curved or bent tip needle commercially available from Johnson & Johnson

Surgical Vision, Inc., Santa Ana, Calif., USA.

In the embodiment of FIG. 1, during the phacoemulsification procedure, a pumping sub-system 24 comprised in a console 28 pumps irrigation fluid from an irrigation reservoir (not shown) to irrigation sleeve 56 to irrigate eye 20. The irrigation fluid is pumped via an irrigation tubing line 43 running from console 28 to an irrigation channel 45 of probe 12, the distal end of the irrigation channel 45 including the fluid pathway in irrigation sleeve 56. Irrigation tubing line 43 is typically flexible and may be prone to collapsing during an occlusion of needle 16. In another embodiment, pumping sub-system 24 may be coupled with or replaced by a gravity fed irrigation source such as a balanced salt solution (BSS) bottle/bag.

Eye fluid and waste matter (e.g., emulsified parts of the cataract) are aspirated via an aspiration channel 47, which extends from the hollow of needle 16 through phacoemulsification probe 12, and then via an aspiration tubing line 46 to a collection receptacle in console 28. The aspiration is effected by a pumping sub-system 26, also comprised in console 28.

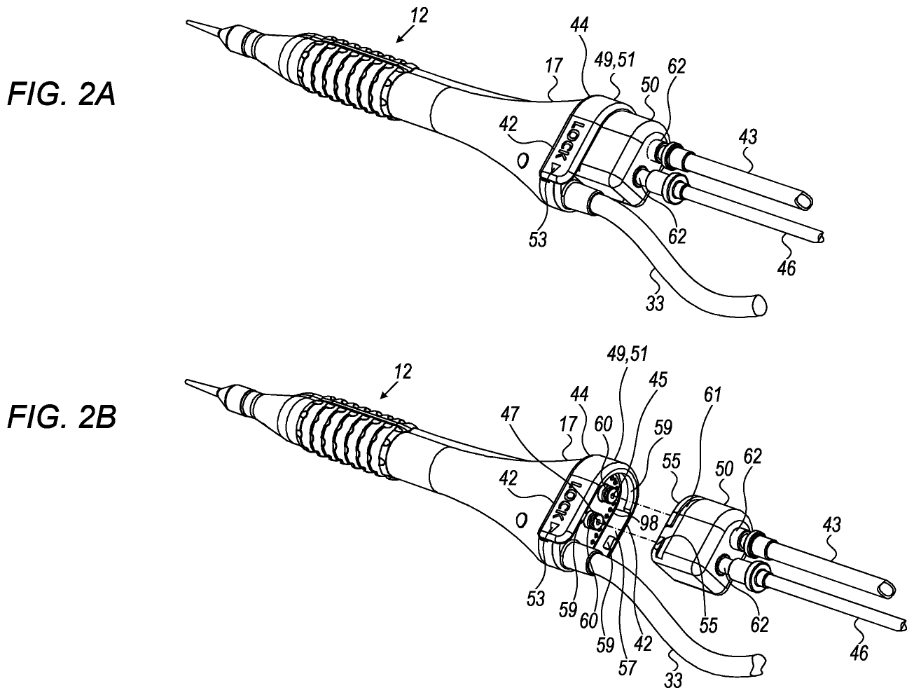

Phacoemulsification probe 12 further comprises a fluid-dynamics cartridge 50, which may comprise one or more valves to regulate the flow of fluid in irrigation channel 45 and/or aspiration channel 47, as well as sensors, described in more detail with reference to FIGS. 2A-B and 3A-C. Cartridge 50 is removably couplable with probe body 17. Part of irrigation channel 45 and aspiration channel 47 is disposed in probe body 17, and part is disposed in cartridge 50.

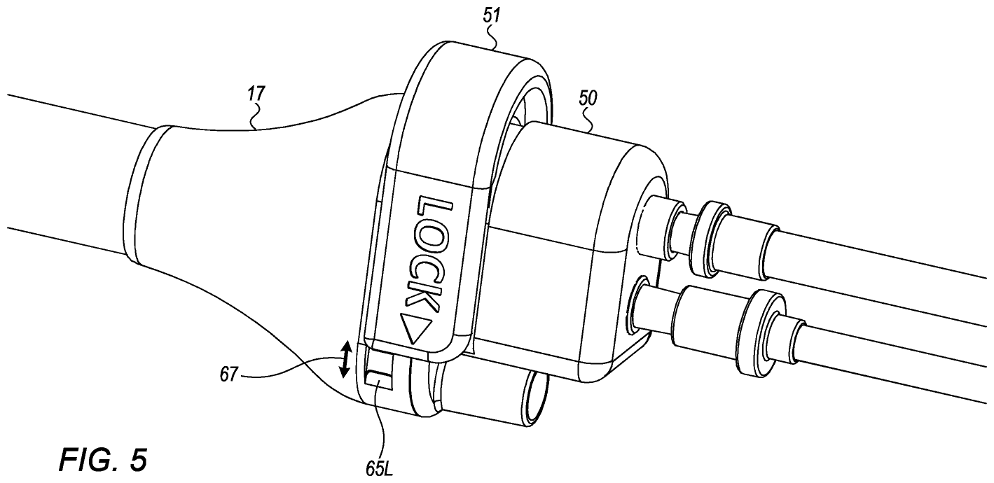

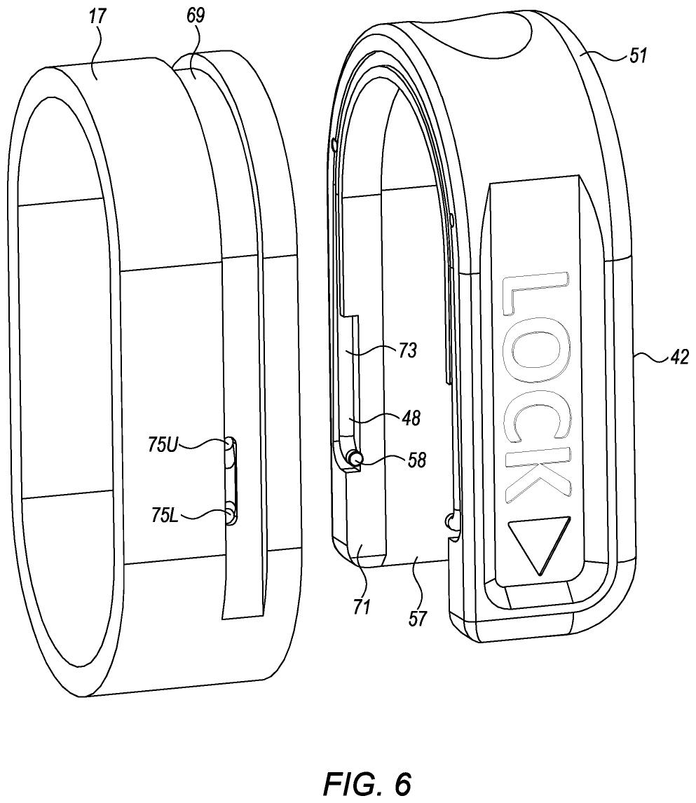

Phacoemulsification probe 12 further comprises other elements, such as a piezoelectric crystal 52 coupled to a horn 54 to drive vibration of needle 16. The piezoelectric crystal is configured to vibrate needle 16 in a resonant vibration mode. The vibration of needle 16 is used to break a cataract into small pieces during a phacoemulsification procedure. Console 28 comprises a piezoelectric drive module 30, coupled with piezoelectric crystal 52, using electrical wiring running in a cable 33. Drive module 30 is controlled by a controller 38 and conveys processor-controlled driving signals via cable 33 to, for example, maintain needle 16 at maximal vibration amplitude. The drive module may be implemented in hardware or software, for example, in a proportional-integral-derivative (PID) control architecture. Controller 38 may also be configured to receive signals from sensors in phacoemulsification probe 12 and, in response to the signals, control one or more valves to regulate the flow of fluid in irrigation channel 45 and/or aspiration channel 47, as described in detail with reference to FIG. 6 of U.S. application Ser. No. 17/240,505. In some embodiments, at least some of the functionality of controller 38 may be implemented using a controller disposed in phacoemulsification probe 12 (e.g., in cartridge 50).

Controller 38 may receive commands via a user interface 40. Such commands may include setting a vibration mode and/or frequency of piezoelectric crystal 52, or setting or adjusting a pumping rate of pumping sub-system 24 or pumping sub-system 26. In some embodiments, user interface 40 and a display 36 may be combined as a single touch screen graphical user interface. In some embodiments, physician 15 uses a foot pedal (not shown) as a means of control. Additionally, or alternatively, controller 38 may receive commands from controls located in a handle 21 of probe 12.

Some or all of the functions of controller 38 may be combined in a single physical component or, alternatively, implemented using multiple physical components. These physical components may comprise hard-wired or programmable devices, or a combination of the two. In some embodiments, at least some of the functions of controller 38 may be carried out by suitable software stored in a memory 35 (as shown in FIG. 1). This software may be downloaded to a device in electronic form, over a network, for example. Alternatively, or additionally, the software may be stored in tangible, non-transitory computer-readable storage media, such as optical, magnetic, or electronic memory.

The system shown in FIG. 1 may include further elements which are omitted for clarity of presentation. For example, physician 15 typically performs the procedure using a stereo microscope or magnifying glasses, neither of which are shown. Physician 15 may use other surgical tools in addition to probe 12, which are also not shown in order to maintain clarity and simplicity of presentation.

### The Phacoemulsification Probe

Reference is now made to FIGS. 2A-B, which are schematic illustrations of phacoemulsification probe 12, in accordance with some embodiments of the present invention.

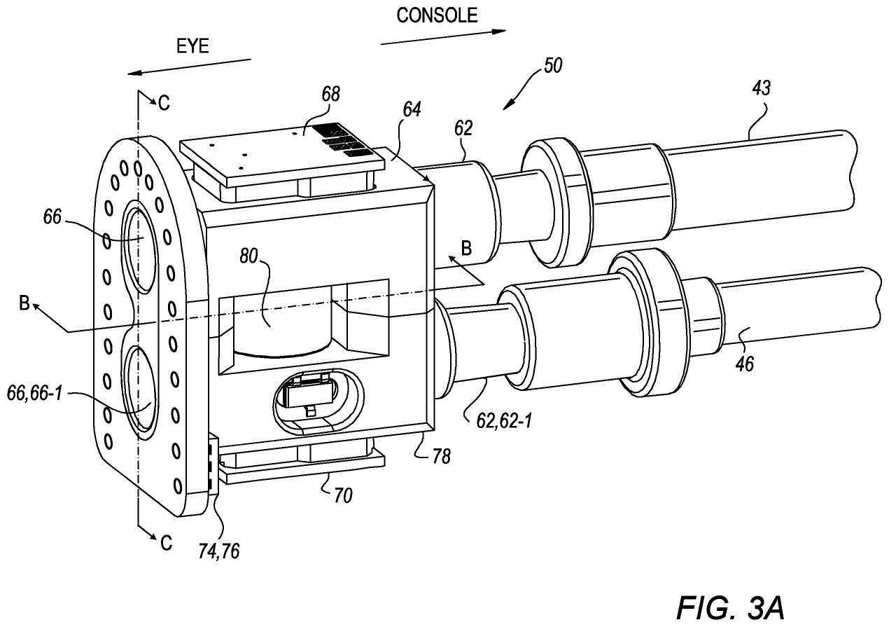

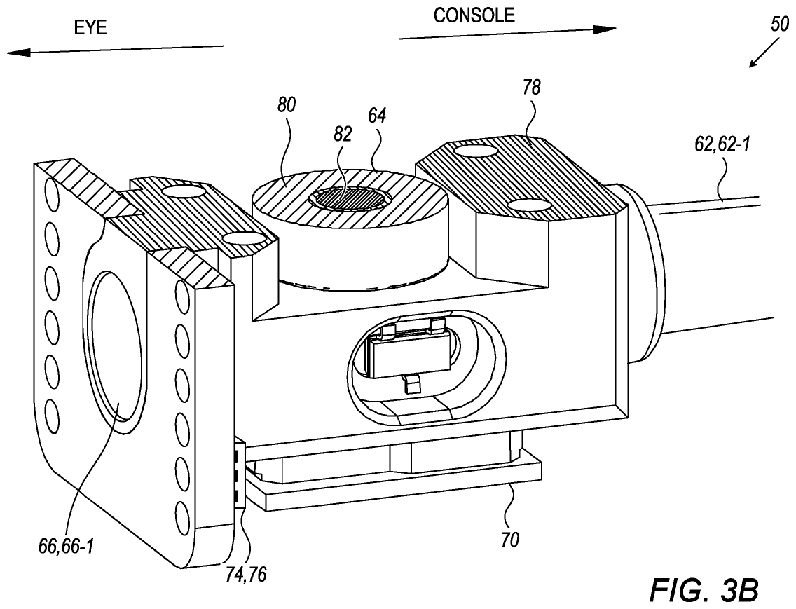

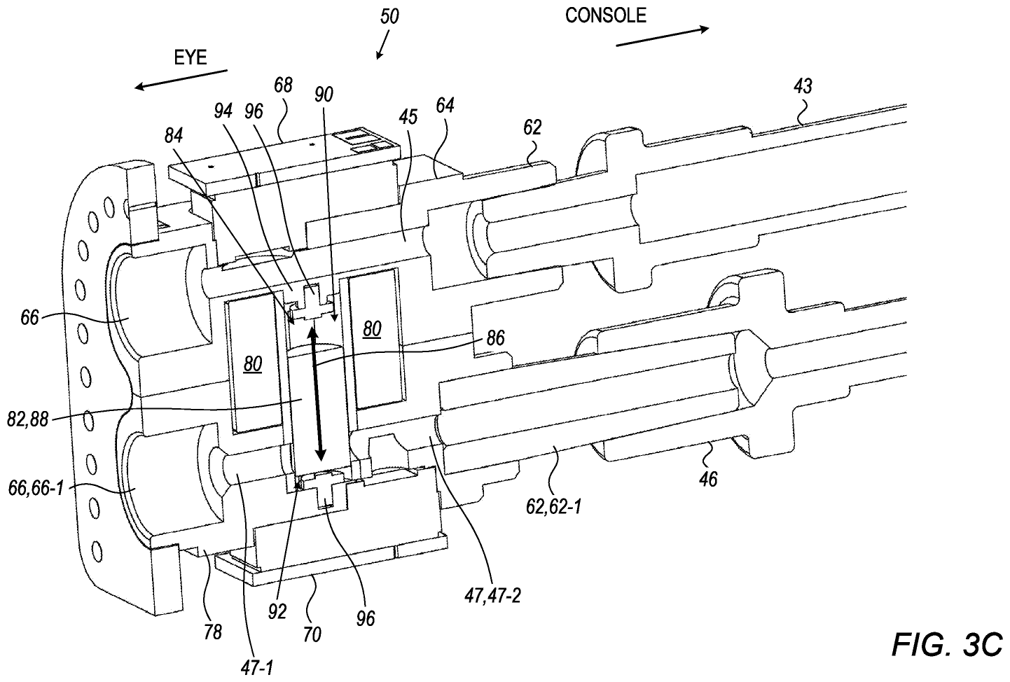

FIG. 2A shows cartridge 50 reversibly coupled with probe body 17 of phacoemulsification probe 12. FIG. 2B shows cartridge 50 disconnected from probe body 17. FIG. 2B further shows connection ports 60 of irrigation channel 45 and aspiration channel 47, disposed at the proximal end of probe body 17, for connecting with corresponding distal ports 66 (FIG. 3A) of cartridge 50 such that the proximal section of irrigation channel 45 within the cartridge is in fluidic communication with the distal section of irrigation channel 45 within the probe body, and the proximal section of aspiration channel 47 within the cartridge is in fluidic communication with the distal section of aspiration channel 47 within the probe body. FIGS. 2A-B also show irrigation tubing line 43 and aspiration tubing line 46 connected to proximal ports 62 of cartridge 50.

In some embodiments, as shown in FIG. 2A, a clip 51 is configured to reversibly couple cartridge 50 with probe body 17 by sliding over the probe body and the cartridge while the cartridge contacts the probe body such that, for each of the channels, the proximal and distal sections of the channel are in fluidic communication with one another. Optionally, clip 51 may comprise a marker 53 and/or explanatory text (such as “LOCK”) that indicates, to the user, the direction in which to slide the clip in order to couple the cartridge with the probe body. As shown in FIG. 2B, to disconnect cartridge 50 from probe body 17, clip 51 may be slid in the opposite direction so as to unlock the cartridge, and the cartridge may then be separated from the probe body.

In some embodiments, clip 51 is U-shaped. For example, clip 51 may comprise two straight arms 42 joined by an arcuate portion 44; alternatively, clip 51 may be entirely arcuate. In other embodiments, clip 51 is elliptical.

Typically, as shown in FIGS. 2A-B, clip 51 is slidably disposed around probe body 17. In such embodiments, the outer surface of cartridge 50 may be shaped to define one or more ridges 55 (e.g., at least two ridges 55 at opposite sides of the cartridge, only one side of the cartridge being shown in FIGS. 2A-B) at the distal end of the cartridge, and the inner surface 57 of clip 51 may be shaped to define one or more protrusions 59 (e.g., two protrusions 59 on different respective arms 42). Clip 51 may thus couple cartridge 50 with probe body 17 by sliding over the probe body and the cartridge until protrusions 59 are aligned with ridges 55, respectively, proximally to the ridges. For example, the outer surface of the cartridge may be shaped to define a groove 61, which is proximal to ridges 55, and protrusions 59 may slide through groove 61 until the protrusions are aligned with the ridges.

(Typically, as assumed throughout the present description, cartridge 50 is coupled with probe body 17 while the distal end of cartridge 50 contacts probe body 17 (e.g., the proximal end of probe body 17), such that clip 51 slides over the distal end of the cartridge. However, it is noted that the scope of the present invention includes embodiments in which the coupling is effected while the proximal end of cartridge 50 contacts the probe body, such that clip 51 slides over the proximal end of the cartridge. Thus, the terms “proximal” and “distal,” as used in the present description, may be used in an illustrative and non-limiting manner. For example, for embodiments in which the proximal end of cartridge 50 contacts probe body 17, ridges 55 may be at the proximal end of the cartridge, and the cartridge may be coupled with the probe body by sliding clip 51 until protrusions 59 are aligned with ridges 55, respectively, distally to the ridges.)

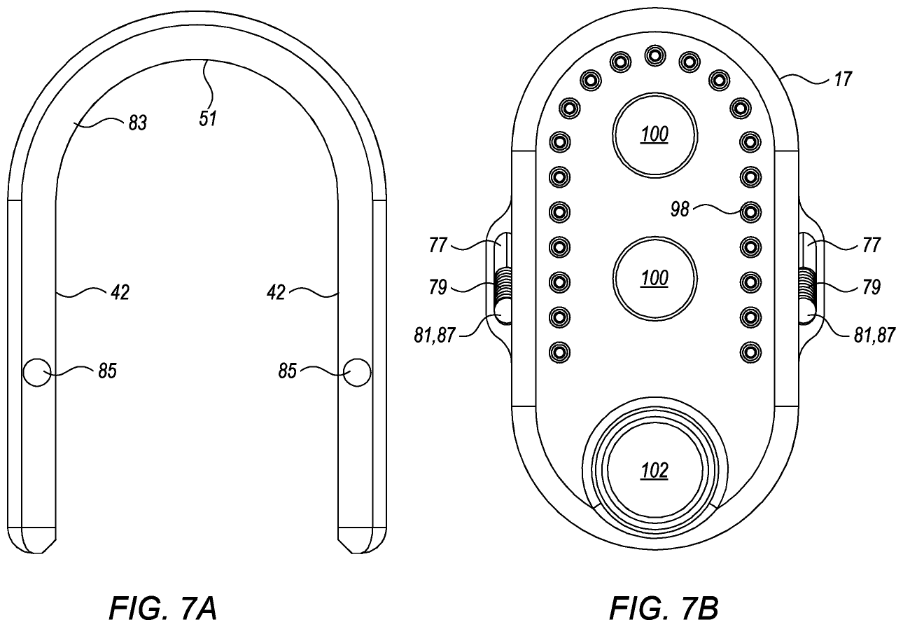

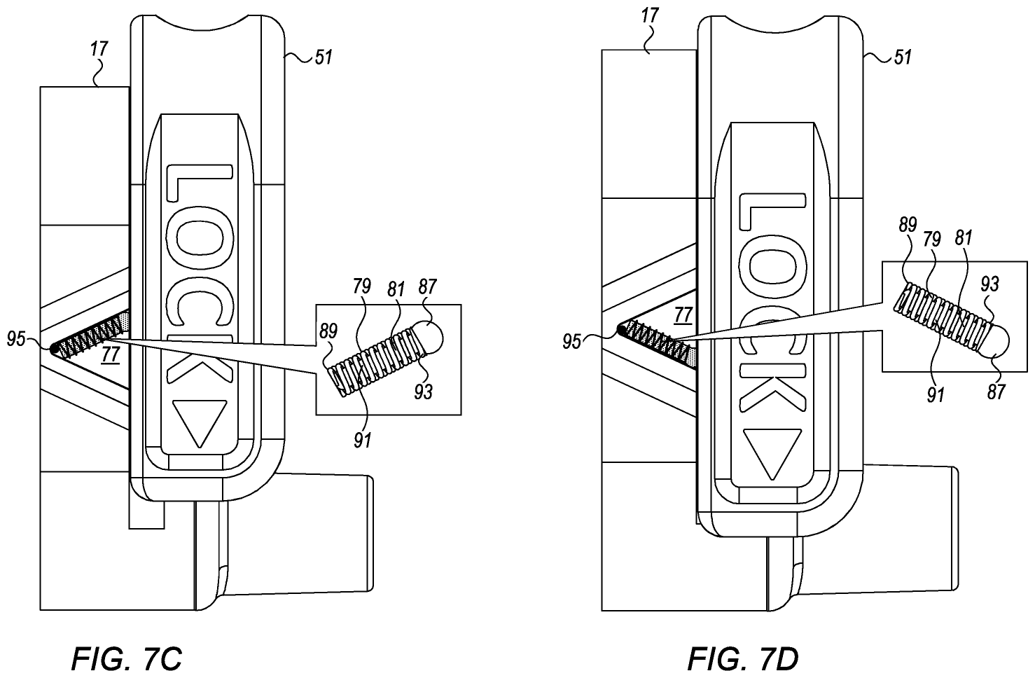

Typically, probe body 17 comprises one or more electrical interfaces, such as pins 98, configured to transfer electricity to cartridge 50 while the cartridge is coupled with the probe body. Thus, electricity transferred to the probe body via cable 33 may power cartridge 50. (Pins 98 are also shown in FIG. 7B, described below.)

Further details regarding the operation of clip 51 are provided below with reference to FIGS. 4-7D.

Reference is now made to FIGS. 3A-C. FIG. 3A is a schematic view of an interior of fluid-dynamics cartridge 50, in accordance with some embodiments of the present invention. FIG. 3B is a cross-section through fluid-dynamics cartridge 50 along line B:B of FIG. 3A, in accordance with some embodiments of the present invention. FIG. 3C is a cross-section through fluid-dynamics cartridge 50 along line C:C of FIG. 3A, in accordance with some embodiments of the present invention.

Typically, phacoemulsification probe 12 comprises a sensor 68 and a sensor 70, each of which may comprise a pressure and vacuum sensor. Probe 12 further comprises a solenoid valve 64, which is traversed by irrigation channel 45 and/or aspiration channel 47. (In the example embodiment shown in FIGS. 3A-C, aspiration channel 47, but not irrigation channel 45, traverses valve 64.) Sensor 68 is coupled with irrigation channel 45. Sensor 70 is coupled with aspiration channel 47 proximally to sensor 68, as shown in FIG. 3C. Sensor 68 and sensor 70 are configured to provide respective signals (e.g., analog signals) indicative of respective fluid metrics (e.g., pressure levels) in irrigation channel 45 and aspiration channel 47.

In some embodiments, as shown in FIGS. 3A-C, cartridge 50 comprises the components mentioned in the preceding paragraph. In such embodiments, solenoid valve 64 may comprise proximal ports 62 for connection to irrigation tubing line 43 and aspiration tubing line 46, and distal ports 66 for connection to connection ports 60 (FIG. 2B). Advantageously, the inclusion of sensors 68 and 70 in cartridge 50 may provide higher sensitivity to local changes in fluid dynamics, and provide a higher degree of control of the pressure in the eye.

Phacoemulsification probe 12 may further comprise a controller 74 configured to receive the aforementioned signals from sensor 68 and/or sensor 70, and, responsively to the received signals, control the fluid connectivity in irrigation channel 45 and/or aspiration channel 47 by selectively opening and closing solenoid valve 64. For example, cartridge 50 may comprise controller 74. In such embodiments, cartridge 50 may further comprise a memory 76 (e.g., an electrically erasable programmable read-only memory (EEPROM)) to store calibration settings and/or a usage counter to help avoid overusing the cartridge. Advantageously, the inclusion of controller 74 in cartridge 50 may facilitate calibrating solenoid valve 64. Additionally, or alternatively, by virtue of being relatively close to sensors 68 and 70, controller 74 may receive any analog signals output by these sensors with less added noise, relative to if the controller were disposed in console 28 (FIG. 1).

Notwithstanding the above, in some embodiments, controller 74 is disposed in console 28, or the functionality of controller 74 is performed by controller 38 (FIG. 1).

As shown in FIG. 3C, aspiration channel 47 includes a section 47-1 coupled with a distal inlet port 66-1 and a section 47-2 coupled with a proximal outlet port 62-1 (as shown in FIG. 3C). Controller 74 is configured to control the fluid connectivity in aspiration channel 47 between inlet port 66-1 and outlet port 62-1 by selectively opening and closing solenoid valve 64 responsively to the fluid metric (e.g., pressure level) in aspiration channel 47. It should be noted that when solenoid valve 64 is closed, sensor 70 shown in FIG. 3C is configured to sense a fluid metric (e.g., pressure level) in section 47-2 between solenoid valve 64 and console 28.

As further shown in FIG. 3C, solenoid valve 64 comprises a valve body 78, a solenoid coil 80, and a plunger 82. Valve body 78 comprises proximal ports 62 and distal ports 66, and is shaped to define a valve cavity 84 having an axis of elongation 86 and configured to provide fluid connectivity between at least one pair of distal and proximal ports (e.g., between inlet port 66-1 and outlet port 62-1).

Solenoid coil 80 is disposed in valve body 78 around valve cavity 84. Plunger 82 comprises a permanent magnet 88 and, optionally, other components, such as a material of low friction that coats or covers permanent magnet 88. Plunger 82 is configured to move back and forth along axis of elongation 86 between a first position 90 and a second position 92 in valve cavity 84, thus selectively allowing or inhibiting fluid connectivity between at least one pair of distal and proximal ports (e.g., between inlet port 66-1 and outlet port 62-1). In some embodiments, permanent magnet 88 is replaced by any suitable magnetic material subjected to a force in a magnetic field, for example, but not limited to, iron, cobalt, nickel, gadolinium, and/or neodymium.

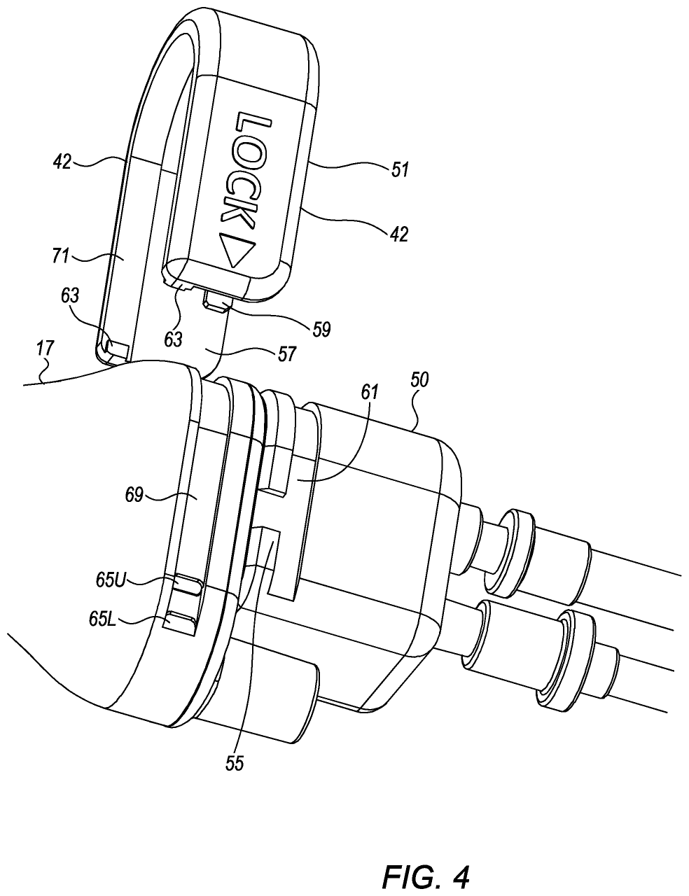

Plunger 82 may have any suitable size, for example, a length in the range of 3 mm to 2 cm (e.g., 6 mm) and a diameter in the range of 1 mm to 1 cm (e.g., 3 mm). Valve body 78 may further comprise a spacer 94, which is described in detail with reference to FIGS. 5A-B of U.S. application Ser. No. 17/240,505.

As the opening and closing of solenoid valve 64 is performed quickly and, sometimes, many times per second, solenoid valve 64 typically comprises one or more shock absorbers 96 configured to soften the striking of plunger 82 against valve body 78 along the axis of elongation 86. For example, solenoid valve 64 may comprise upper and lower shock absorbers 96 (as shown in FIG. 3C) placed at either end of valve cavity 84. Shock absorbers 96 typically comprise, or are generally formed from, a resilient material such as silicone rubber, natural rubber, synthetic rubber, or polyurethane. As shown in FIG. 3C, the upper shock absorber 96 may be disposed within spacer 94.

Controller 74 (FIGS. 3A-B) is configured to apply at least one current to solenoid coil 80 to selectively move plunger 82 between first position 90 and second position 92, and to selectively maintain the plunger in first position 90 or second position 92, as described in detail with reference to FIGS. 5A-B of U.S. application Ser. No. 17/240,505. Further details regarding the structure and operation of phacoemulsification probe 12, and particularly cartridge 50, are also provided in U.S. application Ser. No. 17/240,505.

### Coupling the Cartridge with the Probe Body

As described above with reference to FIGS. 2A-B, clip 51 may couple cartridge 50 with probe body 17 by virtue of protrusions 59 of the clip being aligned with ridges 55 of the cartridge.

Furthermore, typically, probe body 17 and clip 51 are configured to facilitate holding clip 51, alternatingly, in an unlocked position, in which protrusions 59 are misaligned with ridges 55, and a locked position, in which protrusions 59 are aligned with ridges 55. Thus, advantageously, it is relatively easy to use clip 51, in that the clip may simply be slid from one position to the other position. Moreover, as the clip reaches either one of the positions, the user may receive tactile and/or auditory feedback indicating that the clip has reached the position. Several example embodiments providing these advantages are hereby described.

For a first example, reference is now made to FIG. 4, which is a schematic illustration of probe body 17 with cartridge 50 and clip 51, in accordance with some embodiments of the present invention. Reference is further made to FIG. 5, which is a schematic illustration of clip 51 in an unlocked position, in accordance with some embodiments of the present invention.

In some embodiments, inner surface 57 of clip 51 is shaped to define one or more additional protrusions 63, and probe body 17 is shaped to define one or more unlocked-state indentations 65U and one or more locked-state indentations 65L. For example, inner surface 57 may be shaped to define two protrusions 63 at opposite ends of arms 42, and probe body 17 may be shaped to define (i) two unlocked-state indentations 65U on opposite sides of the probe body (one side being shown in FIG. 4), and (ii) two locked-state indentations 65L on opposite sides of the probe body. Protrusions 63 are configured to snap into unlocked-state indentations 65U, respectively, upon clip 51 reaching the unlocked position (as shown in FIG. 5), and to snap into locked-state indentations 65L, respectively, upon clip 51 reaching the locked position.

Thus, as indicated in FIG. 5 by a sliding indicator 67, clip 51 may be slid from one position to the other position. As protrusions 63 enter indentations 65U or 65L, the user may hear a click, and/or feel the protrusions entering the indentations, such that the user knows to stop moving the clip. (It follows that although, for the sake of illustration, FIG. 4 shows clip decoupled from probe body 17, clip 51 is generally not decoupled from probe body 17 when the probe is in use.)

In some embodiments, probe body 17 is shaped to define a groove 69, and inner surface 57 is shaped to define a protruding portion 71 configured to slide within groove 69. In such embodiments, indentations 65U and 65L may be disposed within groove 69, and protrusions 63 may protrude from protruding portion 71.

For another example, reference is now made to FIG. 6, which is a schematic illustration of the proximal end of probe body 17 with clip 51, in accordance with some embodiments of the present invention.

In some embodiments, clip 51 comprises at least one spring 73, and probe body 17 is shaped to define one or more unlocked-state indentations 75U and one or more locked-state indentations 75L. Spring 73 is configured to engage with unlocked-state indentations 75U upon clip 51 reaching the unlocked position, and to engage with locked-state indentations 75L upon clip 51 reaching the locked position.

For example, spring 73 may be generally U-shaped, comprising two arms 48 having respective ends 58 that point inward, toward the probe body. In addition, probe body 17 may be shaped to define (i) two unlocked-state indentations 75U on opposite sides of the probe body (only one side being shown in FIG. 6), and (ii) two locked-state indentations 75L on opposite sides of the probe body. For example, indentations 75U and 75L may be disposed within groove 69, and ends 58 of spring 73 may protrude from protruding portion 71. Spring 73 may thus engage with unlocked-state indentations 75U by virtue of ends 58 springing (or “snapping”) into unlocked-state indentations 75U, and with locked-state indentations 75L by virtue of ends 58 springing into locked-state indentations 75L.

As indicated in FIG. 5 by sliding indicator 67, clip 51 may be slid from one position to the other position. As spring 73 engages with indentations 75U or 75L, the user may hear a click, and/or feel ends 58 springing into the indentations, such that the user knows to stop moving the clip. (It follows that, notwithstanding FIG. 6, clip 51 is generally not decoupled from probe body 17 when the probe is in use.)

For another example, reference is now made to FIG. 7A, which is a schematic illustration of clip 51, in accordance with some embodiments of the present invention. Reference is further made to FIG. 7B, which is a schematic illustration of the proximal end of probe body 17, in accordance with some embodiments of the present invention. (FIG. 7B shows respective apertures 100 through which connection ports 60 (FIG. 2B) pass, along with another aperture 102 into which cable 33 may be inserted.) Reference is further made to FIG. 7C, which is a schematic illustration of clip 51 in an unlocked position, in accordance with some embodiments of the present invention, and to FIG. 7D, which is a schematic illustration of clip 51 in a locked position, in accordance with some embodiments of the present invention.

In some embodiments, as shown in FIG. 7A, the distal face 83 of clip 51 is shaped to define one or more indentations 85. For example, each arm 42 of the clip may be shaped to define a respective indentation 85. In addition, as shown in FIG. 7B, probe body 17 is shaped to define one or more pockets 77; for example, probe body 17 may be shaped to define a respective pocket 77 at each side of the probe body. Probe body 17 comprises one or more pins 81, each pin 81 comprising a front pin-portion 87 disposed within a different respective one of indentations 85 and a back pin-portion 91 (FIGS. 7C-D) disposed within a different respective one of pockets 77. (Front pin-portion 87 may have an enlarged diameter, relative to back pin-portion 91.) Probe body 17 further comprises one or more springs 79, each spring 79 being coupled at a front spring-end 93 (FIGS. 7C-D) to a different respective one of pins 81 and at a back spring-end 89 to the inside of a different respective one of pockets 77. Typically, spring 79 is coiled around back pin-portion 91.

As illustrated in FIGS. 7C-D, springs 79 and pins 81 are configured to rotate within pockets 77 as clip 51 slides between the unlocked position and the locked position. In particular, springs 79 and pins 81 rotate about a lateral axis 95 of probe body 17, which passes from one side of probe body 17 (shown in FIGS. 7C-D) to the other side. The extent of the rotation may be between 10 and 80 degrees, for example, depending on the size of pockets 77.

Spring 79 helps hold clip 51 in the unlocked or locked position, by applying a compressive force that must be overcome in order to slide clip 51 from one position to the other. (In other words, in order to slide clip 51, spring 79 must be compressed.) Moreover, the expansion of spring 79, and/or a sound produced as spring 79 contacts the wall of pocket 77, may indicate to the user that clip 51 has reached the unlocked or locked position.

### Other Embodiments

In some embodiments, clip 51 is slidably disposed around cartridge 50, rather than probe body 17. In such embodiments, the outer surface of the probe body may be shaped to define ridges 55 (FIG. 2B), and clip 51 may be configured to couple cartridge 50 with probe body 17 by sliding over the probe body and the cartridge until protrusions 59 (FIG. 2B) are aligned with ridges 55, respectively, distally to the ridges. Similarly, cartridge 50 and clip 51 may be configured to facilitate holding the clip, alternatingly, in an unlocked position, in which protrusions 59 are misaligned with ridges 55, and a locked position, in which the protrusions are aligned with the ridges. For example:

(i) Cartridge 50 may be shaped to define unlocked-state indentations 65U and locked-state indentations 65L (FIG. 4). Second protrusions 63 (FIG. 4) may thus snap into unlocked-state indentations 65U, respectively, upon clip 51 reaching the unlocked position, and into locked-state indentations 65L, respectively, upon clip 51 reaching the locked position.

(ii) Cartridge 50 may be shaped to define unlocked-state indentations 75U and locked-state indentations 75L (FIG. 6). Spring 73 (FIG. 6) may thus engage with unlocked-state indentations 75U upon clip 51 reaching the unlocked position, and with locked-state indentations 75L upon clip 51 reaching the locked position.

(iii) The proximal face of clip 51 may be shaped to define indentations 85, and cartridge 50 may be shaped to define pockets 77 and may comprise pins 81 and springs 79 (FIGS. 7A-D). Springs 79 and pins 81 may thus rotate within pockets 77 as clip 51 slides between the unlocked position and the locked position.

Alternatively to a phacoemulsification probe, embodiments of the present invention may be implemented with any other medical probe shaped to define at least one fluid channel.

It will be appreciated by persons skilled in the art that the present invention is not limited to what has been particularly shown and described hereinabove. Rather, the scope of embodiments of the present invention includes both combinations and subcombinations of the various features described hereinabove, as well as variations and modifications thereof that are not in the prior art, which would occur to persons skilled in the art upon reading the foregoing description. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A medical probe, comprising:
a probe body shaped to define a distal section of a fluid channel;
a cartridge, which is shaped to define a proximal section of the fluid channel and comprises a valve configured to regulate flow of a fluid through the proximal section of the fluid channel; and
a clip configured to reversibly couple the cartridge with the probe body by sliding over the probe body and the cartridge while the cartridge contacts the probe body such that the proximal section of the fluid channel is in fluidic communication with the distal section of the fluid channel.

2. The medical probe according to claim 1, wherein the clip is U-shaped.

3. The medical probe according to claim 1, wherein the probe body comprises one or more electrical interfaces configured to transfer electricity to the cartridge while the cartridge is coupled with the probe body.

4. The medical probe according to claim 1, wherein the clip is slidably disposed around the probe body.

5. The medical probe according to claim 4,
wherein an outer surface of the cartridge is shaped to define one or more ridges,
wherein an inner surface of the clip is shaped to one or more protrusions, and
wherein the clip is configured to couple the cartridge with the probe body by sliding over the probe body and the cartridge until the protrusions are aligned with the ridges.

6. The medical probe according to claim 5, wherein the probe body and clip are configured to facilitate holding the clip, alternatingly, in an unlocked position, in which the protrusions are misaligned with the ridges, and a locked position, in which the protrusions are aligned with the ridges.

7. The medical probe according to claim 6,
wherein the protrusions are first protrusions,
wherein the inner surface of the clip is further shaped to define one or more second protrusions,
wherein the probe body is shaped to define one or more unlocked-state indentations and one or more locked-state indentations, and
wherein the second protrusions are configured to snap into the unlocked-state indentations, respectively, upon the clip reaching the unlocked position, and to snap into the locked-state indentations, respectively, upon the clip reaching the locked position.

8. The medical probe according to claim 6,
wherein the clip comprises at least one spring,
wherein the probe body is shaped to define one or more unlocked-state indentations and one or more locked-state indentations, and
wherein the spring is configured to engage with the unlocked-state indentations upon the clip reaching the unlocked position, and to engage with the locked-state indentations upon the clip reaching the locked position.

9. The medical probe according to claim 6,
wherein a face of the clip is shaped to define one or more indentations, and
wherein the probe body is shaped to define one or more pockets, and comprises:
one or more pins, each of the pins comprising a front pin-portion disposed within a different respective one of the indentations and a back pin-portion disposed within a different respective one of the pockets; and
one or more springs, each of the springs being coupled at a front spring-end to a different respective one of the pins and at a back spring-end to an inside of a different respective one of the pockets,
the springs and pins being configured to rotate within the pockets as the clip slides between the unlocked position and the locked position.

10. The medical probe according to claim 1, wherein the clip is slidably disposed around the cartridge.

11. The medical probe according to claim 10,
wherein an outer surface of the probe body is shaped to define one or more ridges,
wherein an inner surface of the clip is shaped to one or more protrusions, and
wherein the clip is configured to couple the cartridge with the probe body by sliding over the probe body and the cartridge until the protrusions are aligned with the ridges, respectively.

12. The medical probe according to claim 11, wherein the cartridge and clip are configured to facilitate holding the clip, alternatingly, in an unlocked position, in which the protrusions are misaligned with the ridges, and a locked position, in which the protrusions are aligned with the ridges.

13. The medical probe according to claim 12,
wherein the protrusions are first protrusions,
wherein the inner surface of the clip is further shaped to define one or more second protrusions,
wherein the cartridge is shaped to define one or more unlocked-state indentations and one or more locked-state indentations, and
wherein the second protrusions are configured to snap into the unlocked-state indentations, respectively, upon the clip reaching the unlocked position, and to snap into the locked-state indentations, respectively, upon the clip reaching the locked position.

14. The medical probe according to claim 12,
wherein the clip comprises at least one spring,
wherein the cartridge is shaped to define one or more unlocked-state indentations and one or more locked-state indentations, and
wherein the spring is configured to engage with the unlocked-state indentations upon the clip reaching the unlocked position, and to engage with the locked-state indentations upon the clip reaching the locked position.

15. The medical probe according to claim 12,
wherein a face of the clip is shaped to define one or more indentations, and
wherein the cartridge is shaped to define one or more pockets, and comprises:
one or more pins, each of the pins comprising a front pin-portion disposed within a different respective one of the indentations and a back pin-portion disposed within a different respective one of the pockets; and
one or more springs, each of the springs being coupled at a front spring-end with a different respective one of the pins and at a back spring-end with an inside of a different respective one of the pockets,
the springs and pins being configured to rotate within the pockets as the clip slides between the unlocked position and the locked position.

16. A method, comprising:
bringing a cartridge, which is shaped to define a proximal section of a fluid channel and includes a valve configured to regulate flow of a fluid through the proximal section of the fluid channel, into contact with a probe body of a medical probe, the probe body being shaped to define a distal section of the fluid channel, such that the proximal section of the fluid channel is in fluidic communication with the distal section of the fluid channel; and
reversibly coupling the cartridge with the probe body, by sliding a clip over the probe body and the cartridge while the cartridge contacts the probe body.

17. The method according to claim 16, wherein the clip is U-shaped.

18. The method according to claim 16, wherein the clip is slidably disposed around the probe body.

19. The method according to claim 18,
wherein an outer surface of the cartridge is shaped to define one or more ridges,
wherein an inner surface of the clip is shaped to one or more protrusions, and
wherein sliding the clip comprises sliding the clip over the probe body and the cartridge until the protrusions are aligned with the ridges, respectively.

20. The method according to claim 16, wherein the clip is slidably disposed around the cartridge.
